(19) United States
(12) Patent Application Publication
(10) Pub. No.: US 2025/0134378 A1
(43) Pub. Date: May 1, 2025

(54) AN ULTRA-FLEXIBLE MINIATURE OPTICAL COHERENCE TOMOGRAPHY CATHETER AND IMAGING METHOD FOR ENDOMICROSCOPY OF THE INNER EAR

(71) Applicants: The General Hospital Corporation, Boston, MA (US); Massachusetts Eye and Ear Infirmary, Boston, MA (US)

(72) Inventors: Fang Hou, Boston, MA (US); Alejandro Enrique Ortega, Boston, MA (US); Guillermo Tearney, Cambridge, MA (US); Konstantina Stankovic, Stanford, CA (US)

(21) Appl. No.: 18/835,405

(22) PCT Filed: Jan. 20, 2023
(86) PCT No.: PCT/US23/60953

§ 371 Date: Aug. 2, 2024

Related U.S. Application Data
(60) Provisional application No. 63/305,888, filed on Feb. 2, 2022.
(60) Provisional application No. 63/379,575, filed on Oct. 14, 2022.

(51) Int. Cl. A61B 5/00; A61B 1/00; A61B 1/07; A61B 1/227

(52) U.S. Cl. CPC A61B 5/0066; A61B 1/00133; A61B 1/00194; A61B 1/00195; A61B 1/07; A61B 1/227

(57) ABSTRACT

An imaging tool, including: a drive shaft including a proximal end and a distal end; an optical waveguide including a proximal end and a distal end, the proximal end of the optical waveguide being disposed within the proximal end of the drive shaft, and the distal end of the optical waveguide extending beyond the distal end of the drive shaft; and an optical probe head coupled to the distal end of the optical waveguide.

### CROSS-REFERENCE TO RELATED APPLICATIONS

The present application is based on and claims priority from U.S. Provisional Patent Application No. 63/305,888 filed Feb. 2, 2022, and U.S. Provisional Patent Application No. 63/379,575 filed Oct. 14, 2022, the entire disclosures of which are incorporated herein by reference.

### STATEMENT REGARDING FEDERALLY SPONSORED RESEARCH

N/A

### BACKGROUND

Sensorineural hearing loss (SNHL)—hearing loss caused by defects of the inner ear—affects hundreds of millions of people around the world. The current standard of care for hearing disorders such as SNHL includes diagnostic and treatment procedures. Diagnostic procedures include performing imaging (e.g. CT and/or MRI); assessing behavioral metrics (e.g. pure tone audiometry (Frequency×Level) and/or word recognition ability); and/or assessing physiological metrics (e.g. auditory brainstem response and/or distortion product otoacoustic emissions). Assuming an accurate diagnosis can be made, treatment procedures include providing the subject with a hearing aid and/or a cochlear implant. However, problems with current diagnostic approaches include poor or insufficient imaging resolution, inconsistent measurements, lack of reliability in certain patient populations, and/or lack of sensitivity for certain important types of pathologies. Although being able to obtain high-quality images of the cochlea would provide more accurate diagnoses, there currently are no imaging tools available for imaging the inner ear in humans at the cellular level.

### SUMMARY

Accordingly, various embodiments of the disclosure provide an ultra-flexible miniature optical coherence tomography (OCT) catheter for endomicroscopy of small and/or anatomically complex structures such as the cochlea. Certain embodiments of the catheter include a multi-component sheath structure including a tip of the catheter that is flexible and soft, which permits the catheter to be inserted into structures such as the human cochlea, e.g. through the round window, and navigate turns inside the cochlea safely and ensure that the optical imaging probe can rotate smoothly inside the sheath. The optical probe head is designed such that the components have small size constraints to ensure that the catheter is small enough to be inserted into small and/or complex structures such as the human cochlea and navigate turns inside the structures and to ensure that the imaging resolution and imaging depth meet the requirements for visualization of the cellular structure of the inserted target via OCT technology.

In one embodiment, the disclosure provides an imaging tool, including: a drive shaft including a proximal end and a distal end; an optical waveguide including a proximal end and a distal end, the proximal end of the optical waveguide being disposed within the proximal end of the drive shaft, and the distal end of the optical waveguide extending beyond the distal end of the drive shaft; and an optical probe head coupled to the distal end of the optical waveguide.

In another embodiment, the disclosure provides an imaging method, including: inserting an imaging tool into a luminal sample, the imaging tool including: a drive shaft including a proximal end and a distal end, an optical waveguide including a proximal end and a distal end, the proximal end of the optical waveguide being disposed within the proximal end of the drive shaft, and the distal end of the optical waveguide extending beyond the distal end of the drive shaft, an optical probe head coupled to the distal end of the optical waveguide; and obtaining, using the imaging tool, optical data from the luminal sample.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a diagram of an OCT system that can be used with constructions of the disclosed probe.

FIG. 2A shows a diagram of a protective sheath for a probe according to certain constructions of the present disclosure.

FIG. 2B shows a photograph of a protective sheath for a probe according to certain constructions of the present disclosure, where the inset in the lower right corner highlights the cap; disposed within the protective sheath is a probe such as that shown in FIGS. 3 and 4.

FIG. 3 shows a diagram of an imaging probe according to certain constructions of the present disclosure.

FIG. 4 shows a photograph of an imaging probe according to certain constructions of the present disclosure.

FIG. 5 shows diagrams (panels (A) and (B)) and photographs (panels (C) and (D)) of an optical probe head of an imaging probe according to certain constructions of the present disclosure.

FIG. 6 shows a diagram of an imaging probe disposed within a protective sheath according to certain constructions of the present disclosure.

FIG. 7A shows an apparatus that was used to measure the amount of force needed to deflect a cochlear implant or a silicone sheath containing an 80 μm SMF and FIG. 7B shows graphs of data collected with the apparatus of FIG. 7A indicating that the cochlear implants (CI samples 1-3) required more force to deflect by 20° than the silicone sheath (Our cochlear catheter).

FIG. 8 shows an apparatus for conducting an insertion force test (left panel) on a spiral phantom (first inset, center panel) which mimics the cochlea. The second inset (right panel) shows the rounded cap at the distal end of the protective sheath which helps the sheath advance smoothly through the cochlea.

FIG. 9 shows a graph of the amount of force required to insert either cochlear implants (CI samples 1-3) or a silicone sheath containing an 80 μm SMF (Our cochlear catheter) as a function of depth within the phantom of FIG. 8.

FIGS. 10A and 10B show results of rotation tests to quantify distortion effects associated with the probe at high rotational speeds (3000 rpm).

FIGS. 11A and 11B show a diagram of the optical components of a construction of the disclosed probe (FIG. 11A) which was used to perform optical simulations (FIG. 11B).

FIG. 12A shows a photo and a schematic of an OCT imaging target that was used to characterize the lateral resolution of the disclosed probe, FIG. 12B, 12C show results of characterization of image quality using a construction of the disclosed probe.

FIG. 13 shows an alternative version of an optical probe head which includes an angle polished reflector (R) disposed within a reflective tube (RT) (panels (a) and (b)) where the RT includes an imaging window (panel (c)) and the angled reflective surface is coated (e.g. with a metal such as gold) (panel (d)).

FIG. 14 shows alternative version of an optical probe head with a distal cap that has an optically reflective surface (R) formed therein.

FIG. 15 shows a construction of an optical probe head with a multi-part spacer component shown in an exploded view (top) and an assembled view (bottom).

### DETAILED DESCRIPTION

In accordance with some embodiments of the disclosed subject matter, mechanisms (which can include apparatus, systems, and methods) for providing an ultra-flexible miniature optical coherence tomography catheter for endomicroscopy are provided. Embodiments of the apparatus include an imaging probe disposed within a protective sheath.

In certain embodiments, the disclosure may provide an optical coherence tomography catheter apparatus for endomicroscopy of small and/or anatomically complex structures such as the human cochlea and other parts of the human inner ear which can be used to diagnose conditions such as sensorineural hearing loss (SNHL) and other sensory deficit of the auditory system in humans. Embodiments of the disclosed procedures will allow visualization of cellular and neuronal structures that may be damaged and/or destroyed in patients with SNHL. In various embodiments, embodiments of the disclosed imaging tool can be used to perform 3D imaging of the cochlea prior to cochlear implant insertion surgery, providing 3D reconstruction of the morphology and/or dimensions of the scala tympani so it can facilitate the determination and insertion of the regular cochlear implant. While the cochlea is mentioned herein as an exemplary structure that can be examined using embodiments of the disclosed device, the size and flexibility feature of the device makes it suitable to image various luminal (e.g. hollow or tube-shaped) samples such as other inner ear structures including the vestibular end organs and can be used for imaging through the oval window to image the sensory epithelium of the saccule and/or the utricle or could be used for imaging inside the semicircular canals.

The disclosed device is designed to provide in vivo imaging from inside the cochlea to visualize structures such as the Organ of Corti. The cochlea can be entered via the round window (which is approximately 2 mm) and a probe such as those disclosed herein can be advanced through the scala tympani, which has a diameter ranging from 0.8-1.2 mm. The insertion length of the probe in some embodiments may range from 20-30 mm, where an initial 360° loop through the scala tympani may require a length of approximately 18.5 mm; in other embodiments the insertion length may be such that the probe may be inserted at least 25 mm into the cochlea. The disclosed probe is configured to be capable of conforming to the varying radius of curvatures that are presented within the cochlea, for example from approximately 6 mm in the outer portions down to 2 mm in the inner portions of the scala tympani. The imaging optics of the disclosed device are configured to provide cellular-level imaging data on structures such as hair cells (with dimensions of 7-12 μm×10-70 μm), auditory nerve fibers (with dimensions from 0.5 μm-2 μm).

In various embodiments, meeting the above requirements is made possible by the use of high-resolution OCT (OCT), which has a lateral resolution of 2-3 μm and axial resolution of 1.5-2 μm and a depth of focus (DOF) of approximately 600 μm. To further aid in the insertion of the probe into the deepest portions of the cochlea, the disclosed device includes a rigid portion having a maximum length 1.7 mm or less and a rigid diameter of 600 μm or less. As discussed below, the disclosed probe has mechanical properties similar to those of established cochlear implants, including comparable flexibility and insertion force.

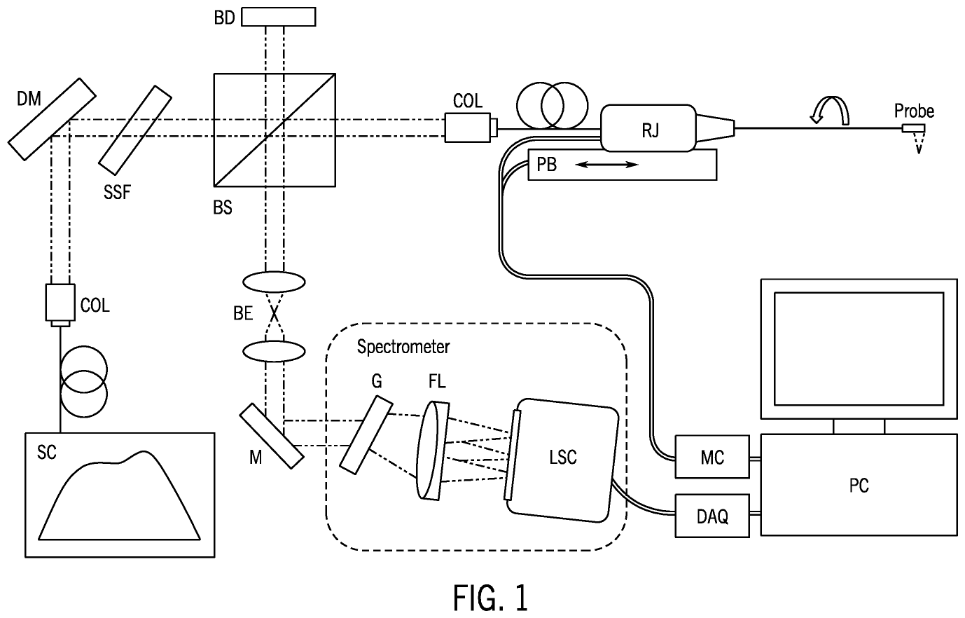

As disclosed in Yin et al. (“μOCT imaging using depth of focus extension by self-imaging wavefront division in a common-path fiber optic probe”, Opt. Express, 2016Mar. 7; 24(5):5555-5564, incorporated herein by reference in its entirety), an OCT probe divides an illumination wavefront into multiple circular propagation modes that are projected as coaxial foci over an extended focal depth range to provide high depth of focus and later resolution in a range of 3-5 μm and axial resolution of approximately 2 μm over an approximately 1 mm depth range. FIG. 1 shows an OCT system that can be used with embodiments of the disclosed OCT imaging probe In FIG. 1, the arrow next to the probe indicate the rotation of the probe and the following abbreviations are applicable: SC, supercontinuum laser; COL, collimator; DM, dichroic mirror; SSF, spectral shape filter; BD, beam dump; BS, beam splitter; BE, beam expander, M, mirror; G, grating; FL, focusing lens, LSC, line scan camera; RJ, rotary junction; PB, pull-back stage; MC, motion controller; and PC, personal computer

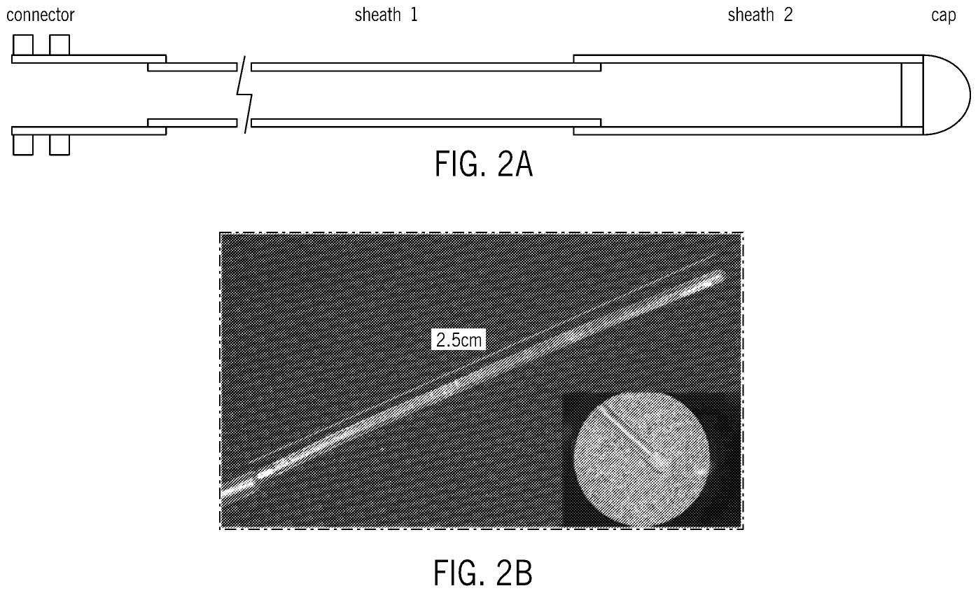

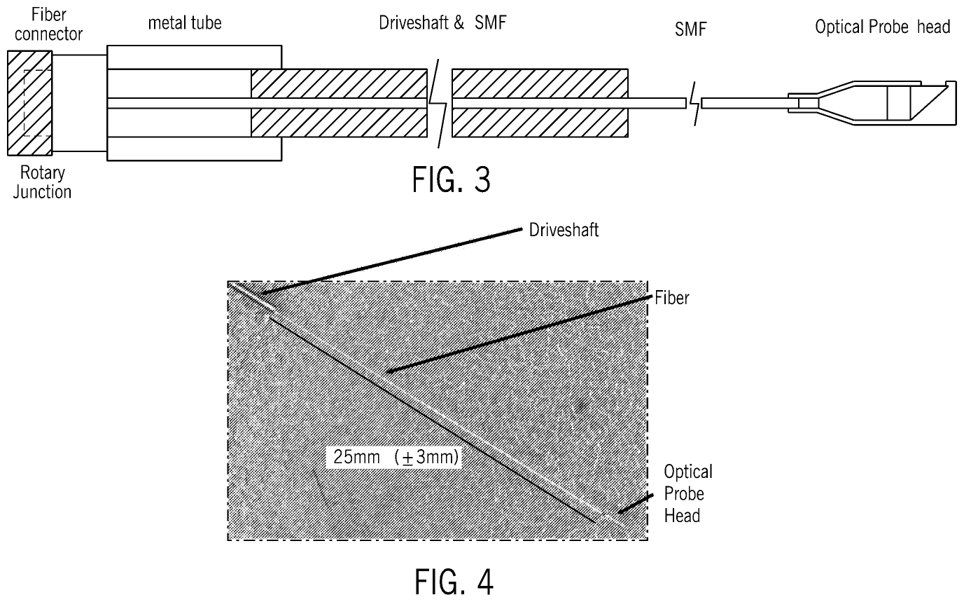

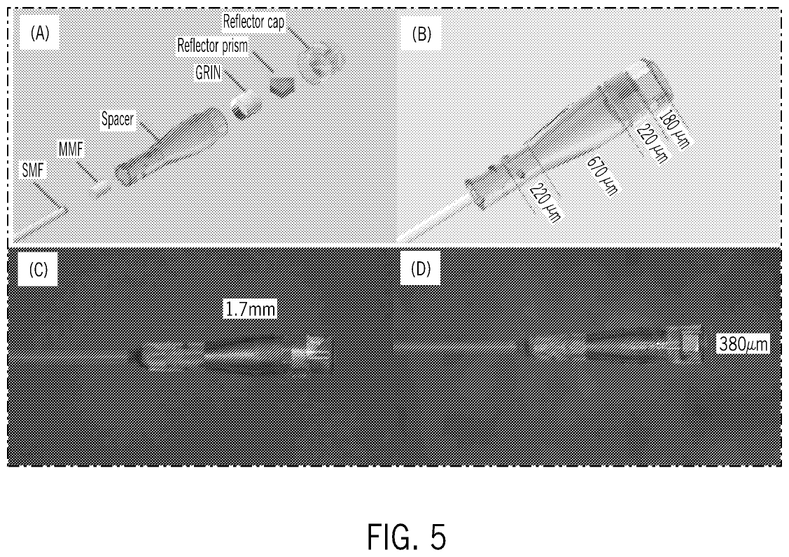

FIG. 2A shows a diagram of a protective sheath for a probe according to certain embodiments of the present disclosure. The protective sheath, which may have a diameter of 600 μm or less, may have as many as four parts, including (from left to right in FIG. 2A): 1. a connector between the sheath and a rotary junction; 2. a low friction plastic sheath (sheath 1) made of a material (e.g. HDPE, Pebax) that has relatively low friction relative to a driveshaft (e.g. having a Teflon coating on the inside surface thereof) rotatably disposed within the sheath; 3. a flexible sheath (sheath 2) made from material (e.g. silicone) that is ultra-flexible and soft; and/or 4. a cap on the silicone sheath that has a specific shape which could reduce insertion trauma of the human cochlea (e.g. a 3D-printed hemispherical cap having a diameter of 600 μm or less). FIG. 2B shows a photograph of a particular embodiment of a protective sheath for a probe according to the present disclosure, where the inset in the lower right corner highlights the cap structure. Disposed within the protective sheath is a probe such as that shown in FIGS. 3 and 4, where the distal end of the drive shaft is shown in the lower left corner of FIG. 2B and the optical probe head is disposed within the distal portion of the sheath, where the “bare” or exposed portion of the optical fiber (i.e. a portion that does not include an overlying drive shaft) plus the optical probe head is at least approximately 2.5 cm/25 mm in length.

FIG. 3 shows a diagram of an imaging probe according to certain embodiments of the present disclosure. The imaging probe may include one or more of the following components (from right to left): 1. an optical probe head; 2. a single-mode fiber which connects the optical probe head to the rotary junction with a fiber connector; 3. a driveshaft that secures the single-mode fiber and provides rotation of the probe; and/or 4. a metal tube that connects the fiber connector and the proximal end of the driveshaft and protects the fiber between them. FIG. 4 shows a photograph of a particular embodiment of an imaging probe according to the present disclosure, where the driveshaft, bare optical fiber, and optical probe head have all been labeled. In this particular example, the bare section of the optical fiber extending beyond the distal end of the driveshaft to the optical probe head (corresponding to the portion labeled “SMF” in FIG. 3) is approximately 25 mm (+/−3 mm), although longer or shorter lengths are possible.

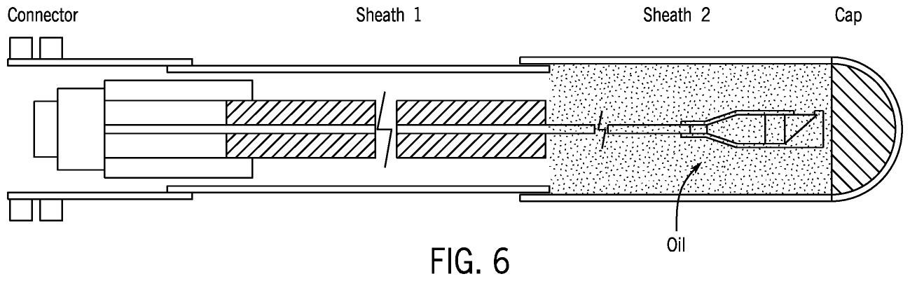

FIGS. 5(A)-5(D) show diagrams (FIGS. 5(A) and 5(B)) and photographs (FIGS. 5(C) and 5(D)) of embodiments of an optical probe head of an imaging probe according to the present disclosure. FIG. 5(A) shows an exploded-view diagram of the optical probe head which depicts (from left/proximal to right/distal) the relative positions of the single mode fiber (SMF), multi-mode fiber (MMF), spacer, GRIN lens (which is inserted into a distal end of the spacer), a reflector prism, and a reflector cap (which houses the reflector prism and attaches to the distal end of the spacer). The spacer may include a cylindrical neck region at its proximal end and a cylindrical distal portion, where the neck region has a smaller diameter than the distal portion, and wherein the two cylindrical portions are joined by a tapered region therebetween. The SMF is inserted into a proximal end of the spacer, in the proximal portion of the narrow neck region, while the MMF is inserted further into the narrow neck region of the spacer, adjacent to a distal end of the SMF (see FIG. 3). FIG. 5(B) shows a side view diagram of the optical probe head in the assembled state, with indications of distances and lengths of various components (discussed further below). FIG. 5(C) shows a photograph of a side view of the optical probe head with a line indicating the total length (1.7 mm) of an embodiment of the probe head and which shows a side view of the prism which highlights the angled reflective surface. FIG. 5(D) shows a photograph of a side view of the optical probe head with a line indicating the width (380 μm) of an embodiment of the probe head and which is rotated approximately 90° (along its long axis) compared to the view in FIG. 5(C) which as a result shows a view of the outer face of the prism and which therefore does not show the angled reflective surface.

Due to the use of lubricant (e.g. mineral oil) in the sheath to facilitate rotation of the optical imaging probe (see below), in various embodiments the prism includes a reflective coating (e.g. is coated with a material such as a metal, for example silver, gold, and/or aluminum) to make the angled surface of the prism reflective independent of whether there is an index of refraction mismatch between the glass and the surrounding media. In various embodiments, the distal surface of the MMF may be angle polished at a certain degree, for example 8°, and the proximal end surface of the GRIN may also be angle polished at the same degree to compensate for the beam direction shift caused by the refraction at the distal surface of the MMF. By adding complementary angle polishing to these components, the back reflection from the two facing surfaces can be suppressed with a result that the SNR of the probe is improved. In certain embodiments, the prism and GRIN lens may be made of materials that are closely matched to one other in refractive index such that the interface between the prism and GRIN lens do not generate a strong back-reflection.

In various embodiments, the optical probe head includes the following parts and may be connected as described herein (see FIGS. 5(A)-5(D)): 1. a single-mode fiber (SMF) may be optically coupled to a multimode fiber (MMF) within the narrow neck region of the spacer; 2. the SMF and MMF may in turn be optically coupled to a hollow tube (e.g. the tapered portion of the spacer) that works to let the light beam expand within its hollow internal space; 3. a GRIN lens may be secured in the distal end of the spacer (e.g. in the distal cylindrical portion of the spacer); 4. a reflector that is disposed at the distal end of the GRIN lens/spacer that reflects the light to the side direction of the catheter. The reflector in this apparatus can be a prism or a glass rod polished at a particular angle (e.g. between) 30°-60° at the distal end; and/or 5. the optical probe may also have a reflector cap which secures the reflector and helps maintain alignment between the reflector and the GRIN lens and spacer.

In various embodiments, the components disclosed herein may have particular size limitations which are selected to achieve the required performance for human intracochlear imaging (see FIG. 5(B)). In one particular embodiment these sizes may include: 1. the MMF may have a core diameter of 20 μm and a cladding diameter of 125 μm or less and the length of the MMF may be 220 μm or less; 2. the spacer tube may include a tapered portion joining a relatively narrower cylindrical neck region at the proximal end and a relatively larger cylindrical distal region, where the outer diameter of the neck region may be 250 μm or less and the outer diameter of the distal region may be 380 μm or less, where the distance between the MMF and GRIN lens (which corresponds to the tapered portion) may be 670 μm; 3. the GRIN lens may have a diameter of 250 μm and the length of the GRIN lens may be 220 μm; 4. the reflective prism may have a length, width, and height of 180 μm; and/or 5. the polished glass rod may have a diameter of 250 μm. In those embodiments in which the neck of the spacer has a diameter of less than 250 μm and/or the distal region has a diameter of less than 380 μm, certain components such as the MMF, the GRIN lens, and the polished glass rod may be made proportionately smaller as well so that the components complement one another.

The sizes are features of the optical probe head design and may have small tolerance ranges. Various embodiments of the optical probe head may have different dimensions which may depend in part on considerations such as the anatomical structure into which a probe containing the optical probe head will be inserted.

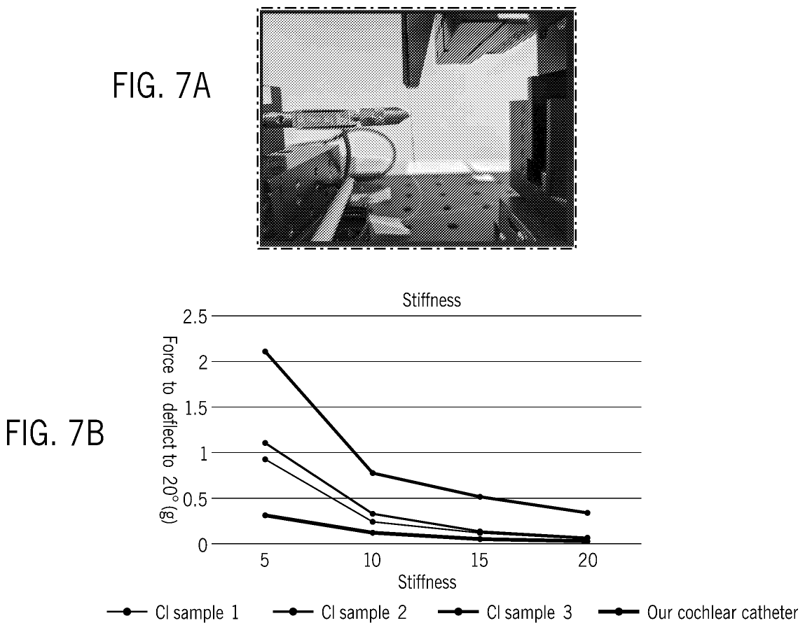

FIG. 6 shows a diagram of an imaging probe disposed within a protective sheath according to certain constructions of the present disclosure. During the assembly of the catheter, lubricant (e.g. mineral oil; see FIG. 6) may be injected into the silicone sheath to ensure the rotation of the optical imaging probe inside of the sheath. The single-mode fiber may extend from the driveshaft for a particular length, which may be 25 mm (+/−3 mm) in certain embodiments of the apparatus (see FIG. 2B). Including this “bare” section of the SMF between the distal end of the driveshaft and the spacer increases the overall flexibility of the probe by permitting the portion near the end of the probe to flex without the constraint of the relatively stiff drive shaft.

In use, light from the micro-optical coherence tomography imaging system propagates into the optical imaging probe through the connector between the probe and the rotary junction. When the light beam propagates in the core of the MMF, a few light transmission modes will be generated because of the reflection of the core-cladding interface of the MMF. After the beam expands within the spacer and is focused onto the GRIN lens and reflected onto the reflector, the light will be delivered to the tissue of interest inside the human cochlea, passing through the lubricant and the protective sheath. The few-mode light beam will be focused along the optical axis in the region of interest and will generate an extended focusing depth range. The back reflection from the illuminated region will be collected by the same optical imaging probe as it propagates back to the micro-optical coherence tomography imaging system and generates images of the tissue.

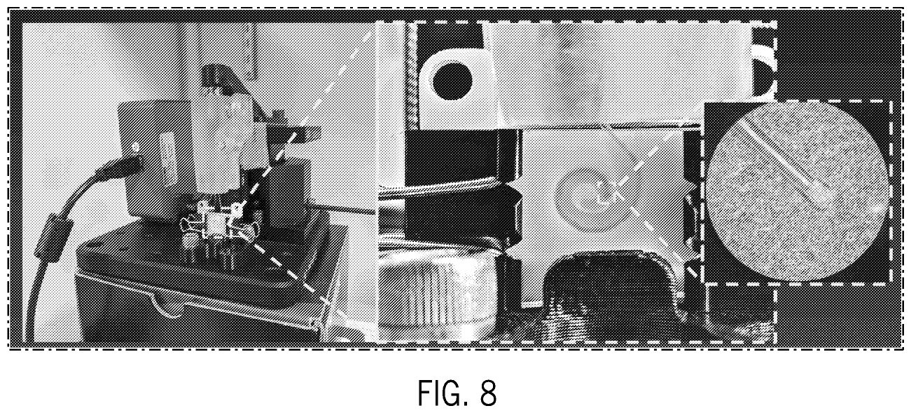

In various embodiments, the catheter is designed to be flexible. The imaging depth of the probe may be 500 μm with an average material imaging resolution between 4-5 μm. A stiffness test was conducted to measure the flexibility of the probe compared to several cochlear implant samples. Cochlear implants were used as points of reference when assessing the properties of the present device because the implanted portion of the cochlear implant system is already established as being sufficiently small and stiff to permit insertion in the cochlear space. Thus, if the present device has properties that are comparable to those of known cochlear implants, which are themselves capable of being inserted into the cochlea, then it should be possible to insert the present device into the cochlea without too much difficulty. Accordingly, an apparatus (FIG. 7A) was used to measure the amount of force needed to deflect a cochlear implant (CI samples 1-3) or a silicone sheath containing an 80 μm SMF (Our cochlear catheter) by 20°. As seen in FIG. 7B, substantially less force was required to deflect the silicone sheath by 20° than any of the cochlear implant devices that were tested, indicating that the disclosed device is more than sufficiently flexible to be insertable into the cochlea.

Flexibility (stiffness) of cochlear implants has been shown to correlate to the trauma caused by the insertion, with a more flexible implant causing less trauma. Various embodiments of the disclosed device have similar or smaller bending stiffness to commercial cochlear implants and thus are similarly suitable for insertion into the inner ear. Measured data of the bending stiffness value of embodiments of OCT catheter and commercial cochlear implants (shown in FIG. 7B) demonstrates the force needed to deflect the sample to 30 degrees at different distance from the clamped point. Bending stiffness can be calculated from the measured data using the following equation:

$$E \times I = \frac{F \times {L}^{3}}{3 ω}$$

Where E×I is the bending stiffness (E: Modulus of elasticity, I: Moment of inertia). F is the force applied to the sample at the point with a distance of L to the fixation point, and ω is the deflection distance. The measured bending stiffness of certain embodiments of the probe at the flexible distal portion (i.e. the ˜25 mm extending part with the bare fiber part of the probe and the flexible silicone sheath) may fall in the range of 4.41*10⁻⁸ Nm² to 6.81*10⁻⁸ Nm². For comparison, the bending stiffness of certain cochlear implant samples falls in the range of 1.12*10⁻⁸ Nm² to 7.58*10⁻⁸ Nm². Thus, in some embodiments the distal portion of the disclosed imaging tool has a bending stiffness of less than 7.58*10⁻⁸ Nm².

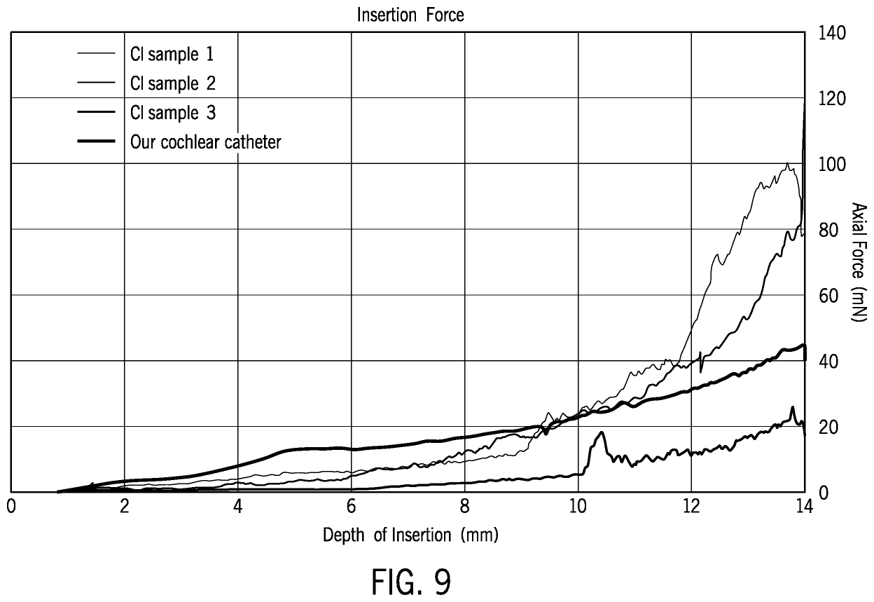

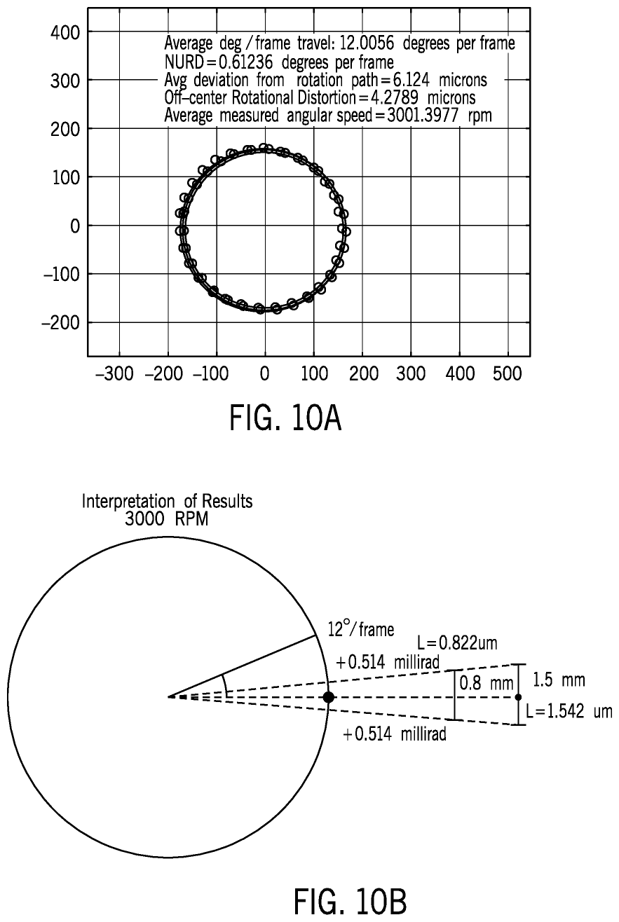

FIG. 8 shows an apparatus for conducting an insertion force test (left panel) on a spiral phantom (first inset, center panel) which mimics the cochlea. The second inset (right panel) shows the rounded cap at the distal end of the protective sheath which helps the sheath advance smoothly through the cochlea. FIG. 9 shows a graph of the amount of force required to insert either cochlear implants (CI samples 1-3) or a silicone sheath containing an 80 μm SMF (Our cochlear catheter) as a function of depth within the phantom. As seen in FIG. 9, while the cochlear catheter required an increasing amount of force as it traveled deeper into the phantom, it still required less force than two out of the three cochlear implants that were tested, indicating that it is capable of being inserted into the cochlea without requiring application of an unusually large amount of force.

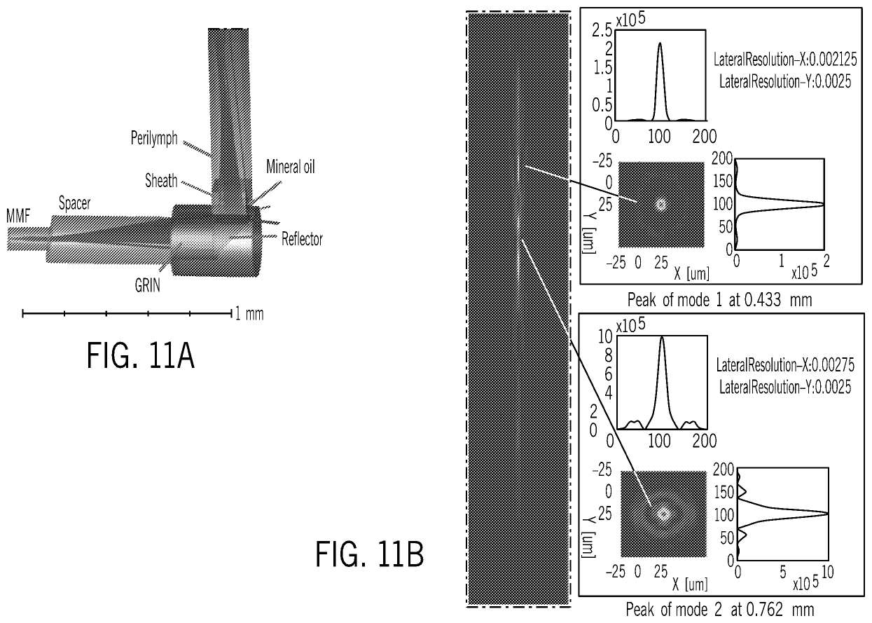

FIGS. 10A and 10B show results of rotation tests to quantify distortion effects associated with the probe at high rotational speeds (3000 rpm). FIG. 10A shows the path of rotation for the probe which collects 30 frames per rotation. Based on the data, it was determined that the average degrees of rotation of travel per frame was 12.0056° with a standard error of 0.514 milliradians; the non-uniform rotational distortion (NURD) was 0.61236° per frame; the average deviation from the rotational path was 6.124 μm; the off-center rotational distortion was 4.2789 μm; and the average measured angular speed was 3001.3977 rpm. FIG. 10B shows a diagram interpreting the results of FIG. 10A where the consequences of the standard error of 0.514 milliradians are shown at points 0.8 mm and 1.5 mm from the probe are depicted.

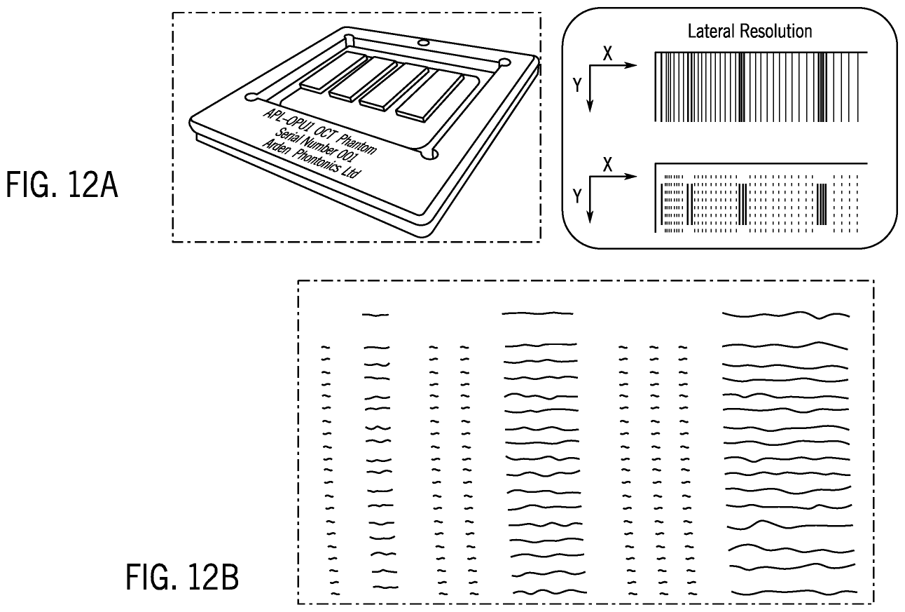

FIGS. 11A and 11B show a diagram of the optical components of a construction of the disclosed probe (FIG. 11A) which was used to perform optical simulations (FIG. 11B). The optical components include a SMF (with a 3.5 μm core and 80 μm cladding), a MMF (with a 20 μm core and 125 μm cladding), a spacer (a 3D printed tube), a 250 μm GRIN lens, and a reflector including a 180 μm prism. The diagram in FIG. 11A also shows the locations of other optically-relevant components including the sheath, the layer of mineral oil within the sheath, and the perilymph adjacent to the sheath. The depth of focus (DOF) of the setup of FIG. 11A is approximately 600 μm, the imaging range is approximately 0.42 mm-0.98 mm, and the average lateral resolution is approximately 4.6 μm. FIG. 11B (left vertical panel) shows a simulation of a beam emitted from the optical setup of FIG. 11A (with the beam having an increasing distance from the probe going from top to bottom in the diagram) along with two cross-sectional views of the beam at different points corresponding to the peaks of mode 1 at 0.433 mm and mode 2 at 0.762 mm (upper and lower right panels, respectively).

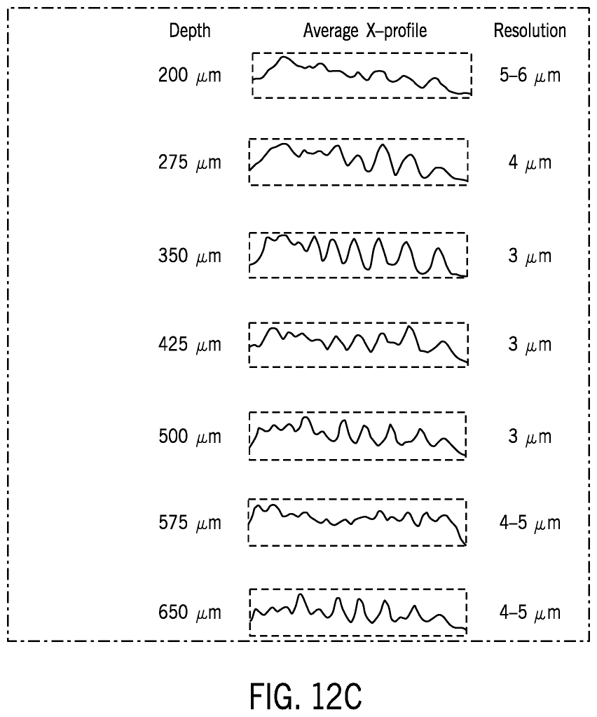

FIGS. 12A, 12B, and 12C show results of characterization of image quality using a construction of the disclosed probe. FIG. 12A shows an OCT phantom (left panel) and the lateral resolution grids of the phantom (right panel). FIG. 12B shows a close-up image corresponding to a depth of 425 μm. The first column of FIG. 12C shows a series of images from the phantom of FIG. 12A with particular images being encircled by dashed boxes corresponding to various depths as indicated in the “Depth” column, along with an average X-profile through the respective image. The last column of FIG. 12C shows the determined resolution at the particular depth, which was best (i.e. has the lowest value, 3 μm) in the 350-500 μm range, which is close to the target lateral resolution of 2-3 μm.

Accordingly, disclosed herein are embodiments of an ultra-flexible miniature micro-OCT catheter that can be inserted into the human cochlea through the round window and can be used to obtain cellular level micro-OCT images. Characterization results disclosed herein show that the catheter has comparable mechanical properties, including flexibility and insertion force, to commercially available cochlear implants. Imaging of OCT phantoms shows that a catheter which includes the disclosed micro-OCT system can achieve ˜2 μm axial resolution and <5 μm lateral resolution within an approximately 500 μm focusing range.

### Variations

The following are variations on one or more of the components disclosed herein.

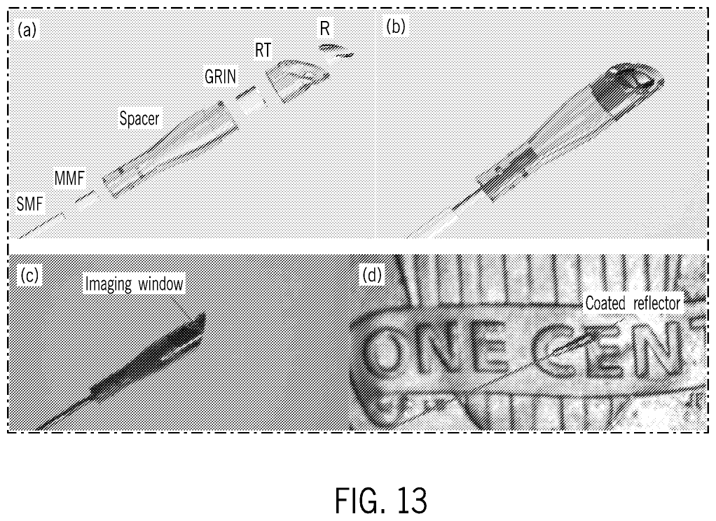

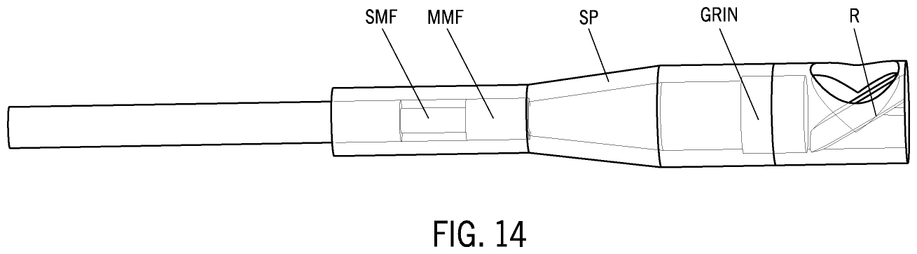

In some embodiments, a polished glass rod may be used as a reflector in place of the reflector prism shown in FIG. 5(A). As shown in FIGS. 13(a) and 13(b), in one particular embodiment the optical probe head is similar to that shown in FIGS. 5(A) and 5(B) except that in place of a reflector prism and reflector cap, the probe head instead includes an angle-polished reflector (R) which is placed in a reflector tube (RT). In one procedure for forming the embodiment shown in FIGS. 13(a) and 13(b), a 250 μm glass rod was first inserted into the 3D printed RT after which the rod was cleaved slightly longer than the RT. The RT with the glass rod was aligned and attached to the main body of the probe at the distal end (connected to the GRIN lens). After this, the glass rod required a manual cleave, a vertical polish, and then a 45° polish. On the RT, an eye-shaped opening (FIG. 13(c)) was included on the side opposite the angled surface to allow the light beam to come out from the probe and focus on the tissue. As noted above, mineral oil is used between the probe and sheath to make sure the probe can rotate smoothly within the silicone sheath. The mineral oil also acts as an index matching oil, preventing aberrations of the light beam that can lead to poor imaging. However, given that the optics are bathed in mineral oil, it is not feasible to use total internal reflection of the polished surface to direct light to the side of the probe and therefore the polished surface is coated (e.g. with a metal such as silver, gold, and/or aluminum) in order to be reflective (FIG. 13(d)). In another embodiment, the optical probe head has a 3D printed cap that includes an optically reflective surface (R) formed therein (FIG. 14).

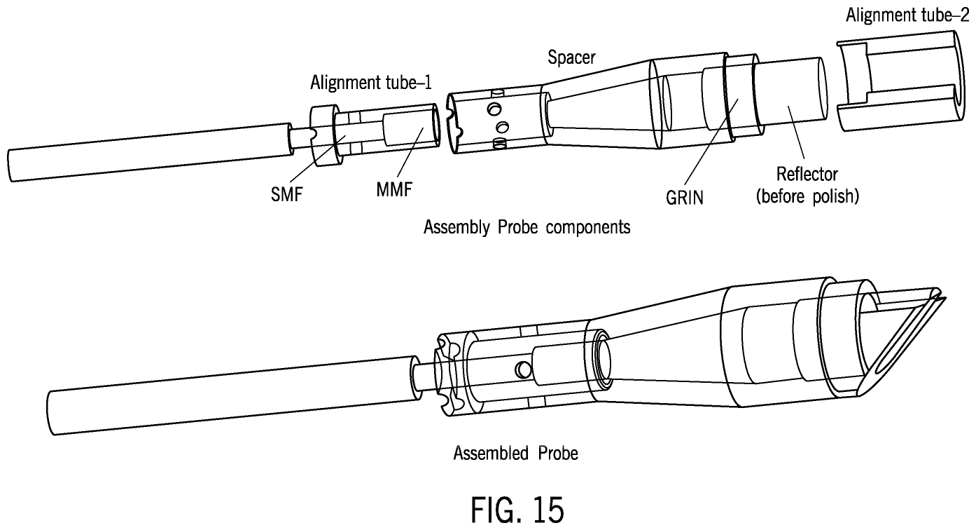

FIG. 15 shows an embodiment of an optical probe head with a multi-part spacer component shown in an exploded view (top) and an assembled view (bottom). In this embodiment, the spacer is fabricated as two separate components in order to improve the alignment of the optical components. A first alignment tube (Alignment tube-1) includes the SMF and MMF and this first alignment tube is inserted into a complementary opening in the proximal portion of the spacer (FIG. 15, top panel). A second alignment tube (Alignment tube-2) houses the reflector and brings the reflector into position so that it is adjacent the GRIN lens. As shown in the top panel of FIG. 15, in this embodiment a glass rod is inserted into the second alignment tube prior to polishing and the alignment tube and glass rod are polished as a single unit to produce the reflector component shown in the assembled form in the bottom panel of FIG. 15. In this particular embodiment, the first and second alignment tubes and the spacer are 3D printed.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. An imaging tool, comprising:
a drive shaft including a proximal end and a distal end;
an optical waveguide including a proximal end and a distal end,
the proximal end of the optical waveguide being disposed within the proximal end of the drive shaft, and
the distal end of the optical waveguide extending beyond the distal end of the drive shaft; and
an optical probe head coupled to the distal end of the optical waveguide.

2. The imaging tool of claim 1, wherein the distal end of the optical waveguide extending beyond the distal end of the drive shaft comprises a bare portion of the optical waveguide, and
wherein the bare portion of the optical waveguide is disposed within a flexible sheath.

3. The imaging tool of claim 2, further comprising a lubricant disposed within the flexible sheath.

4. The imaging tool of claim 3, further comprising a rounded cap coupled to a distal end of the flexible sheath.

5. The imaging tool of claim 3, further comprising a rotary junction coupled to the proximal end of the optical waveguide and the proximal end of the drive shaft,
wherein the optical waveguide and the optical probe head are rotatably disposed within the flexible sheath.

6. The imaging tool of claim 1, wherein the optical probe head comprises a spacer at a proximal end thereof, wherein the distal end of the optical waveguide is coupled to a proximal end of the spacer.

7. The imaging tool of claim 6, wherein the spacer comprises a conical shape having a narrow proximal end adjacent the optical waveguide.

8. The imaging tool of claim 7, wherein the narrow proximal end has a diameter of 250 μm or less and wherein the spacer comprises a wide distal end having a diameter of 380 μm or less.

9. The imaging tool of claim 7, wherein the optical probe head further comprises a reflector to reflect light from the optical waveguide away from a central axis of the optical waveguide.

10. The imaging tool of claim 9, wherein the optical probe head further comprises a reflector cap at a distal end thereof, and wherein the reflector is disposed within the reflector cap.

11. The imaging tool of claim 9, wherein the optical probe head further comprises a reflector cap at a distal end thereof, and wherein the reflector cap comprises an optically reflective surface formed therein.

12. The imaging tool of claim 11, wherein the reflector cap is 3D printed.

13. The imaging tool of claim 10, wherein the reflector comprises a prism, and
wherein a reflective surface of the prism comprises a reflective coating comprising gold.

14. The imaging tool of claim 10, wherein the reflector comprises a polished glass rod, and
wherein a reflective surface of the prism polished glass rod comprises a metallic coating.

15. The imaging tool of claim 13, further comprising a GRIN lens disposed within a distal end of the spacer adjacent to the prism.

16. The imaging tool of claim 7, wherein the optical waveguide comprises a single mode fiber,
wherein the narrow proximal end of the spacer comprises a multimode fiber disposed therein adjacent to the distal end of the optical waveguide.

17. The imaging tool of claim 16, wherein the multimode fiber has a core diameter of 20 μm, a cladding diameter of 125 μm or less, and a length of 220 μm or less.

18. The imaging tool of claim 1, wherein the optical probe head has a length of 1.7 mm which defines a maximum rigid length of the imaging tool.

19. The imaging tool of claim 4, wherein the rounded cap has a diameter of 600 μm or less.

20. The imaging tool of claim 2, wherein the bare portion of the optical waveguide is at least 25 mm in length.

21. The imaging tool of claim 2, wherein the drive shaft is disposed within a low friction sheath adjacent to the flexible sheath.

22. The imaging tool of claim 21, wherein an inside surface of the low friction sheath comprises a Teflon coating.

23. The imaging tool of claim 2, wherein the sheath has a diameter of 600 μm or less, wherein the optical probe head has a length of 1.7 mm or less which defines a maximum rigid length of the imaging tool, and wherein a distal portion of the imaging tool can be inserted to a depth of at least 25 mm into the cochlea.

24. The imaging tool of claim 23, wherein the distal portion of the imaging tool can be inserted into a curved sample having a radius of curvature of at least 2 mm.

25. The imaging tool of claim 23, wherein the distal portion of the imaging tool comprises the bare portion of the optical waveguide, the optical probe head, and the flexible sheath.

26. The imaging tool of claim 2, wherein a distal portion of the imaging tool has a bending stiffness of less than 7.58*10⁻⁸ Nm², wherein the distal portion of the imaging tool comprises the bare portion of the optical waveguide, the optical probe head, and the flexible sheath.

27. An imaging method, comprising:
inserting an imaging tool into a luminal sample, the imaging tool comprising:
a drive shaft including a proximal end and a distal end,
an optical waveguide including a proximal end and a distal end,
the proximal end of the optical waveguide being disposed within the proximal end of the drive shaft, and
the distal end of the optical waveguide extending beyond the distal end of the drive shaft, and
an optical probe head coupled to the distal end of the optical waveguide; and
obtaining, using the imaging tool, optical data from the luminal sample.

28. The method of claim 27, wherein inserting an imaging tool into a luminal sample further comprises:
inserting the imaging tool into the inner ear of a subject.

29. The method of claim 28, wherein inserting an imaging tool into the inner ear of a subject further comprises:
inserting the imaging tool into at least one of the cochlea, the saccule, the utricle, or the semicircular canal of the subject.

30. The method of claim 27, wherein the distal end of the optical waveguide extending beyond the distal end of the drive shaft comprises a bare portion of the optical waveguide,
wherein the bare portion of the optical waveguide is disposed within a flexible sheath, and
wherein an imaging tool into a luminal sample further comprises:
inserting the flexible sheath containing the bare portion of the optical waveguide into the luminal sample.

31. The method of claim 30, wherein the sheath has a diameter of 600 μm or less,
wherein the optical probe head has a length of 1.7 mm or less which defines a maximum rigid length of the imaging tool, and
wherein inserting the flexible sheath containing the bare portion of the optical waveguide into the luminal sample further comprises:
inserting the flexible sheath containing the bare portion of the optical waveguide to a depth of at least 25 mm into the luminal sample.

32. The method of claim 31, wherein inserting the flexible sheath containing the bare portion of the optical waveguide into the luminal sample further comprises:
inserting the flexible sheath containing the bare portion of the optical waveguide into the luminal sample comprising a curved portion having a radius of curvature of at least 2 mm.

33. The method of claim 27, wherein obtaining optical data from the luminal sample further comprises:
obtaining three-dimensional imaging data from the luminal sample to identify at least one of a morphology or a dimension of the scala tympani to facilitate insertion of a cochlear implant.
